(19) United States
(12) Patent Application Publication
(10) Pub. No.: US 2026/0224448 A1
(43) Pub. Date: Aug. 6, 2026

(54) SOLID COMPOSITION FOR AQUEOUS, VISCOUS FORMULATION

(71) Applicant: SPECIALTY OPERATIONS FRANCE, Lyon (FR)

(72) Inventors: Varun TRIPATHI, Vadodara (IN); Vishal JAVIA, Vadodara (IN); Nikolay CHRISTOV, Newtown, PA (US)

(21) Appl. No.: 19/154,875

(22) PCT Filed: Feb. 26, 2024
(86) PCT No.: PCT/EP2024/054785

§ 371 Date: Aug. 8, 2025

(30) Foreign Application Priority Data
Mar. 8, 2023 (IN) 202341015425

(51) Int. Cl. A61K 8/02; A61K 8/36; A61K 8/44; A61K 8/46; A61Q 19/10

(52) U.S. Cl. CPC A61K 8/0225; A61K 8/36; A61K 8/44; A61K 8/466; A61Q 19/10; A61K 2800/30

(57) ABSTRACT

The present invention relates to a solid composition for forming an aqueous, viscous liquid.

### TECHNICAL FIELD

The present invention relates to a solid composition for forming an aqueous, viscous liquid.

### TECHNICAL BACKGROUND

Solid compositions comprising surfactants are used in many industrial applications. One important example is the use for forming aqueous liquids with a sufficient viscosity and structure that can be used as a cleanser or as a personal care composition.

Solid compositions of the prior art for such uses commonly contain an anionic surfactant and a thickener. U.S. Pat. No. 4,330,438 describes a powder shampoo concentrate comprising a mixture of an anionic surfactant and a gum.

However, conventional solid compositions of the prior art require the use of thorough mixing under high shear rates to provide structures that are evenly distributed and free of lumps. Thus, such solid compositions often provide unsatisfactory results when mixed by the layman or consumer at home and not under laboratory settings.

Therefore, it was an object of the present invention to provide a solid composition that provides a uniform mixture as an aqueous, viscous liquid with excellent structure and viscosity, when combined with water, even in absence of a thickener.

### SUMMARY OF THE INVENTION

These objects have surprisingly been solved by a solid composition comprising:
(a) at least one anionic surfactant,
(b) at least one amphoteric surfactant or at least one acyl taurate, and
(c) at least one alkali metal salt of a free fatty acid,
   wherein the wt.-ratio of (a) the at least one anionic surfactant to (b) the at least one amphoteric surfactant or the at least one acyl taurate ranges from 1:3 to 1:0.1, and the wt.-ratio of (a) the anionic surfactant to (c) the alkali metal salt of a free fatty acid ranges from 1:1.5 to 1:0.01, with the proviso that (a) the at least one anionic surfactant and (b) the at least one acyl taurate are different from each other.

It has surprisingly been found that the solid compositions of the present invention, when mixed with water, lead to an aqueous liquid with excellent viscosity. In particular, it has been surprisingly found that the solid compositions of the present invention, when mixed with water, lead to an aqueous liquid with excellent viscosity without adding (i.e., in the absence of) thickeners. That is, the instant solid compositions can make viscous aqueous liquids without the need of thickeners when mixed or dispersed in water. In addition to the excellent rheology properties, an aqueous liquid can be obtained that is a uniform mixture and is free of turbidities.

In another aspect, the present invention relates to an aqueous liquid comprising the solid composition of the present invention and an aqueous solvent, wherein the aqueous liquid has a viscosity of 200 cP to 25,000 cP.

In another aspect, the present invention relates to a process of forming the above-mentioned aqueous liquid comprising mixing the solid composition of the present invention with an aqueous solvent.

In another aspect, the present invention relates to the use of the solid composition of the present invention for forming the aqueous liquid.

In yet another aspect, the present invention relates to the use of the aqueous liquid of the present invention as a cleaning composition, personal care composition like body wash and/or facial cleanser and/or hand wash and/or hair wash (shampoo) and/or skin/hair conditioner and/or multipurpose skin/hair cleanser, disinfection composition, floor cleanser, toilet bowl cleanser, surface cleansing composition like glass, and/or metal and/or plastic and/or ceramic.

### DETAILED DESCRIPTION OF THE INVENTION

The following definitions are relevant in connection with the embodiments of the present invention.

The meaning of the term “comprising” is to be interpreted as encompassing all the specifically mentioned features as well optional, additional, unspecified ones, whereas the term “consisting of” only includes those features as specified. Therefore, “comprising” includes as a limiting case the composition specified by “consisting of”.

The term “wt. %” refers to the amount of the respective ingredient by weight based on the total amount of the composition, unless noted otherwise.

As used herein, the singular forms “a”, “an”, and “the” include both singular and plural referents unless the context clearly dictates otherwise.

The term “aqueous liquid” refers to a liquid that comprises at least 25 wt. % water. The aqueous liquid is also known as a cleanser liquid. It is preferred that the aqueous liquid is completely liquid. The aqueous liquid may also comprise undissolved parts of the solid composition of the present invention and therefore also encompasses a dispersion.

Preferred embodiments according to the invention are defined hereinafter. The preferred embodiments are preferred alone or in combination. Further, it is to be understood that the following preferred embodiments refer to all aspects of the present invention, i.e. the solid composition, the aqueous liquid, the process of forming the aqueous liquid, the use of the solid composition as well as the use of the aqueous liquid.

In an embodiment, the present invention relates to a solid composition comprising:
(a) at least one anionic surfactant,
(b) at least one amphoteric surfactant or at least one acyl taurate, and
(c) at least one alkali metal salt of a free fatty acid,
   wherein the wt.-ratio of (a) the at least one anionic surfactant to (b) the at least one amphoteric surfactant or the at least one acyl taurate ranges from 1:3 to 1:0.1, and the wt.-ratio of (a) the at least one anionic surfactant to (c) the at least one alkali metal salt of a free fatty acid ranges from 1:1.5 to 1:0.01, with the proviso that (a) the at least one anionic surfactant and (b) the at least one acyl taurate are different from each other.

In an embodiment, the at least one anionic surfactant is not an acyl taraute. In an embodiment, the present invention relates to a solid composition comprising:
(a) at least one anionic surfactant,
(b) at least one amphoteric surfactant, and
(c) at least one alkali metal salt of a free fatty acid,
   wherein the wt.-ratio of (a) the at least one anionic surfactant to (b) the at least one amphoteric surfactant ranges from 1:3 to 1:0.1, and the wt.-ratio of (a) the at least one anionic surfactant to (c) the at least one alkali metal salt of a free fatty acid ranges from 1:1.5 to 1:0.01.

In an embodiment, the present invention relates to a solid composition comprising:
(a) at least one anionic surfactant,
(b) at least one acyl taurate, and
(c) at least one alkali metal salt of a free fatty acid,
   wherein the wt.-ratio of (a) the at least one anionic surfactant to (b) the at least one acyl taurate ranges from 1:3 to 1:0.1, and the wt.-ratio of (a) the at least one anionic surfactant to (c) the at least one alkali metal salt of a free fatty acid ranges from 1:1.5 to 1:0.01, with the proviso that (a) the at least one anionic surfactant and (b) the at least one acyl taurate are different from each other.

The use of at least one acyl taurate has the additional advantage that the presence of the at least one acyl taurate and the absence of an amphoteric surfactant reduces the hygroscopicity of the solid composition. This allows for easier packaging without the need to for moisture-tight packaging. In addition, the solid composition of the present invention comprising at least one acyl taurate has improved flexibility regarding the particle size of the solid composition and the solid composition may be in powder form. In a preferred embodiment, the wt.-ratio of (a) the at least one anionic surfactant to (b) the at least one amphoteric surfactant or the at least one acyl taurate ranges from 1:2 to 1:0.1, and the wt.-ratio of (a) the at least one anionic surfactant to (c) the at least one alkali metal salt of a free fatty acid ranges from 1:1 to 1:0.1.

In an embodiment, the wt.-ratio of (b) the at least one amphoteric surfactant or the at least one acyl taurate to (c) the at least one alkali metal salt of a free fatty acid ranges from 2:1 to 1:0.05, preferably from 1:1 to 1:0.1.

In an embodiment, the solid composition comprises the at least one anionic surfactant in an amount of 10 wt. % to 80 wt. %, preferably 20 wt. % to 70 wt. %, and more preferably 30 wt. % to 60 wt. %.

In an embodiment, the composition comprises the at least one amphoteric surfactant or the at least one acyl taurate in an amount of 5 wt. % to 70 wt. %, more preferably 10 wt. % to 60 wt. %, and more preferably 12 wt. % to 55 wt. %.

In an embodiment, the composition comprises the at least one metal salt of a free fatty acid in an amount of 0.1 wt % to 40 wt. %, preferably 0.5 wt. % to 30 wt. %, more preferably 1 wt. % to 25 wt. %, even more preferably 8.5 wt. % to 25 wt. %.

Thus, in a particularly preferred embodiment, the solid composition comprises the at least one anionic surfactant in an amount of 30 wt. % to 60 wt. %, the at least one amphoteric surfactant or the at least one acyl taurate in an amount of 12 wt. % to 55 wt. %, and the at least one metal salt of a free fatty 20 acid in an amount of 1 wt. % to 25 wt %.

Suitable surfactants are described in “Surfactants and Interfacial Phenomena”, M. Rosen et al., Wiley, 2012 and “Applied Surfactants”, T. Tadros, Wiley-VCH, 2005. In an embodiment, the at least one anionic surfactant is selected from alkyl glycinates, alpha olefin sulphonates, alkyl sulfosuccinates, alkyl sulfo acetates, alkyl lactylates, alkyl carboxylates, alkyl ether carboxylates, acyl glycinates, acyl taurates, acyl taurates acyl sarcosinates, alkyl sulfates, alkyl ether sulfates, acyl isethionates, acyl alaninates, and mixtures of two or more thereof. In a preferred embodiment, the anionic surfactant is an alpha olefin sulphonate and/or an alkyl glycinates, more preferably a C₁₂-C₁₈ alpha olefin sulphonate and/or C₁₂-C₁₈ alkyl glycinates and/or sodium lauroyl sulfosuccinate and/or sodium coco-sulphate, even more preferably a C₁₄ alpha olefin sulphonate. In a preferred embodiment, the at least one anionic surfactant comprises sodium C₁₄ alpha olefin sulphonate and/or sodium cocoyl glycinate and/or sodium lauroyl glycinate and/or sodium cocoyl methyl taurate and/or sodium lauroyl methyl taurate and/or sodium cocoyl glutamate and/or sodium lauroyl glutamate and/or potassium salts of these surfactants. In a preferred embodiment, the anionic surfactant is not a C₁₈ acyl taurate, such as sodium methyl oleoyl taurate.

The term “alkyl” preferably refers to a C₄-C₂₆ alkyl, more preferably to a C₁₂-C₁₈ alkyl. The term “acyl” preferably refers to a C₄-C₂₆ acyl, more preferably to a C₁₂-C₁₈ acyl. The term “alpha olefin” preferably refers to a C₄-C₂₆ alpha olefin, more preferably to a C₁₂-C₁₈ alpha olefin.

In certain embodiments, it is particularly preferred that the at least one acyl taurate is a C₁₄ to C₃₆ acyl taurate, is preferably a C₁₆ to C₃₀ acyl taurate, and more preferably is a C₁₈ to C₂₄ acyl taurate. It is even more preferred that the at least one acyl taurate is a C₁₈ acyl taurate, such as oleoyl taurate (i.e. sodium methyl oleoyl taurate).

Anionic surfactants dissociate in water to yield an anion and a cation in water. In a solid composition, the at least one anionic surfactant is a charge-neutral compound in the form of the free acid or a cosmetically acceptable salt thereof. Non-limiting examples of cosmetically acceptable salts are salts comprising ammonium, sodium, potassium and triethanolammonium. The term “acyl glycinate” refers to a compound of formula R¹NHCH₂CO₂M wherein R¹ is an acyl and M is selected from H or a water-soluble cation, such as ammonium, sodium, potassium and triethanolammonium. The term “acyl taurate” refers to a compound of formula R¹N(R²)CH₂CH₂SO₃M wherein R¹ is an acyl and Mis selected from H or a water-soluble cation, such as ammonium, sodium, potassium and triethanolammonium, and wherein R² is an alkyl, preferably methyl, The term “acyl glutamate” refers to a compound of formula R¹NH(COOM₁)CH₂CH₂COOM₂ wherein R¹ is an acyl and M¹ and M² are independently selected from H and a water-soluble cation, such as ammonium, sodium, potassium and triethanolammonium.

Amphoteric surfactants have an anionic and a cationic functional group. In an embodiment, the at least one amphoteric surfactant is selected from alkylamidopropyl betaines, alkyl betaines, alkylamidopropyl hydroxysultaines, alkyl hydroxysultaines, alkyl amphoacetates, alkyl amphodiacetates, alkyl amphopropionates, alkyl amphodipropionates, alkyl amine N-oxides, alkyldimethylamine N-oxides, alkylamidopropylamine N-oxides, and mixtures of two or more thereof. In a preferred embodiment, the at least one amphoteric surfactant is an alkylamidopropyl hydroxysultaine. In a particularly preferred embodiment, the at least one amphoteric surfactant is cocamidopropyl hydroxysultaine.

In an embodiment, the at least one alkali metal salt of the free fatty acid is selected from alkali metals salts of C₆ to C₂₄ fatty acids, preferably from potassium cocoate, potassium laurate, potassium oleate, potassium ricinoleate, sodium ricinoleate, sodium palmate, sodium palm kernelate, sodium laurate, sodium caprylate, sodium palmitate, potassium stearate, and mixtures of two or more thereof. In an embodiment, the at least one alkali metal salt of a free fatty acid also comprises the free fatty acid itself.

In an embodiment, the solid composition comprises:
(a) at least one cleansing surfactant,
(b) at least one primary structurant, and
(c) at least one secondary structurant,
   wherein the wt.-ratio of (a) the at least one cleansing surfactant to (b) the at least one primary structurant ranges from 1:3 to 1:0.1, and the wt.-ratio of (a) the at least one cleansing surfactant to (c) the at least one acidic surfactant ranges from 1:1.5 to 1:0.01. The cleansing surfactant is commonly acceptable personal care cleansing surfactants which are generally of anionic nature. The primary structurant is either an amphoteric surfactant or a long carbon chain taurate or methyl taurate surfactant. The secondary structurant is an acidic surfactant, which is defined as anionic surfactant with at least one carboxylic acid group in dissociated (salt form) or undissociated state.

In a particularly preferred embodiment, the solid composition comprises
(a) at least one anionic surfactant,
(b) at least one amphoteric surfactant or at least one acyl taurate, and
(c) at least one alkali metal salt of a free fatty acid,
   wherein the wt.-ratio of (a) the at least one anionic surfactant to (b) the at least one amphoteric surfactant or the at least one acyl taurate ranges from 1:3 to 1:0.1, and the wt.-ratio of (a) the at least one anionic surfactant to (c) the at least one alkali metal salt of a free fatty acid ranges from 1:1.5 to 1:0.01, wherein the solid composition comprises the at least one anionic surfactant in an amount of 30 wt. % to 60 wt. %, the at least one amphoteric surfactant in an amount of 12 wt. % to 40 wt. %, and the metal salt of a free fatty acid in an amount of 1 wt. % to 15 wt %, and wherein the at least one anionic surfactant is an alpha olefin sulphonate, an acyl taurate and/or an alkyl glycinate, the at least one amphoteric surfactant is an alkylamidopropyl hydroxysultaine and the at least one alkali metal salt of a free fatty acid is an alkali metal salt of a C₆ to C₂₄ fatty acid, with the proviso that (a) the at least one anionic surfactant and (b) the at least one acyl taurate are different from each other.

The composition of the present invention can further comprise a nonionic surfactant, preferably selected from alkanolamides, fatty alcohol, fatty alcohol ethoxylates, alkylphenol ethoxylates, glycerol fatty acid amides, glycerol fatty acid esters, sorbitol fatty acid esters and mixtures of two or more thereof. Non-limiting examples of fatty alcohol ethoxylates are diethylene glycol monododecyl ether (C12E2), triethylene glycol monododecyl ether (C12E3), tetra ethylene glycol monododecyl ether (C12E4), pentaethylene glycol monododecyl ether (also referred to as C12E5), hexaethylene glycol monododecyl ether (C12E6), heptaethylene glycol monododecyl ether (C12E7), and octaethylene glycol monodecyl ether (C12E8). Non-limiting examples of alkylphenol ethoxylates are Triton X-100® (octyl phenol ethoxylate) and Nonidet P-40® (4-nonylphenol ethoxylate). Non-limiting examples of alkanolamides are cocamide monoethanolamine and cocamide diethanolamine. Non-limiting examples of glycerol fatty acid esters are glycerol monostearate and glycerol monolaurate. Non-limiting examples of sorbitol fatty acid esters are sorbitan monolaurate, sorbitan monostearate and sorbitan tristearate.

The solid composition of the present invention may further comprise an electrolyte, preferably an inorganic salt, such as NaCl, KCl, CaCl₂, NH₄Cl, Na₂SO₄. As can be seen from the Examples, the presence of an inorganic salt, such as sodium chloride, leads to a further increase of viscosity when the solid composition is mixed with water. Thus, in a preferred embodiment, the electrolyte is an alkali metal salt, preferably selected from NaCl, KCl, Na₂SO₄, K₂SO₄, Na₃PO₄, K₃PO₄, and mixtures of two or more thereof. In an embodiment, the solid composition comprises the electrolyte is present in an amount of 0.5 wt. % to 30 wt. %, preferably of 1 wt. % to 20 wt. %, more preferably of 1.5 wt. % to 18 wt. %.

The solid composition can further comprise a stabilizing agent. The stabilizing agent can be selected from the group of benzoic acid and salts thereof, benzyl alcohol, chlorocresol, paraben esters, sorbic acid and salts thereof, sodium metabisulfite, chelating agents, and mixtures of two or more thereof.

The solid composition can further comprise an acidifying agent. Without being bound to theory, it is believed that the use of an acidifying agent leads to the formation of free fatty acids from the at least one alkali metal salts of free fatty acid, which enhances the viscosity upon dilution with water. Thus, it has surprisingly been found that the solid composition of the present invention further comprising an acidifying agent can form viscous liquid cleanser with acidic pH upon dilution with water.

The acidifying agent is preferably a solid compound. In certain embodiments, the acidifying agent can be an organic acid, inorganic acid, or mixtures thereof, and preferably can be at least one organic acid. In certain preferred embodiments, the acidifying agent is selected from citric acid, tartaric acid, fumaric acid, malic acid, ascorbic acid, glucono delta-lactone, salicylic acid, and mixtures thereof. In a particularly preferred embodiment, the acidifying agent comprises citric acid.

In an embodiment, the ratio of (c) the at least one alkali metal salt of a free fatty acid to the acidifying agent ranges from 10:1 to 1:10, from 5:1 to 1:5 or from 3:1 to 1:3. Preferably, the solid composition comprises 0.1 to 20 wt. % acidifying agent, more preferably 1 to 15 wt. %. In certain embodiments, the solid composition can comprise 0.1 to 10 wt. %, more preferably from 0.5 to 5 wt. % of the acidifying agent.

The solid composition can further comprise vitamins, flavonoids, terpenes, saccharides, plant extracts, and/or mixtures of two or more thereof. The solid composition can further comprise chelating agents, preservatives, emollients, hair conditioners, skin conditioners, humectants, opacifiers, bulking agents (fillers), polymers, fragrants, antioxidants, pro-vitamins, dyes, abrasives, UV absorbers, anti UV agents, pigments, synthetic and natural benefit actives like anti dandruff agent, anti seborrheic dermatitis agents, anti psoriasis agents, anti inflammatory agents, skin colouring agents, hair colouring agents, melanin regulators, skin lightening, bleaching agents, anti wrinkle agents, anti ageing agents, keratolytic agents, refreshing agents, cooling agents, essential oils, antimicrobial agents, and/or hair transformation, such as straightening, weaving, colouring, and bleaching agents.

The solid composition is not limited in its form or shape. However, for ease of handling, the solid composition can be in the form of a powder or in the form of a granulate or in the form of a continuous solid, e.g. in the form of a brick or pellet.

Even though the use of thickeners can be avoided by the solid composition of the present invention, the solid composition may comprise thickener to further increase the viscosity properties. Therefore, in an embodiment the solid composition comprises a thickener. The term “thickener” refers to polymeric thickeners, such as anionic or nonionic water soluble or swellable polymers, having high molecular weight and which are able to substantially increase the viscosity of water-based cleansing formulations. Thus, the term thickeners does not encompass electrolyte salts, such as NaCl. Thickeners can be selected from guar gum, xanthan gum, gellan gum, carrageenan gum, cellulose gum, polyacrylates, copolymers of methyl vinyl ether and maleic acid, poly(vinylpyrrolidone), silica, clays, such as sodium montmorillonite, starch, polyvinyl alcohol, pectin, agar, gelatin, collagen, hyaluronic acid, alginate, chitosan, cassia gum, locust bean gum, and mixtures of two or more thereof. The term cellulose gum refers to polycarboxymethyl cellulose ether of cellulose. It is to be understood that the terms guar gum, xanthan gum, gellan gum, carrageenan gum, cassia gum, locust bean gum as well as cellulose gum also encompass derivatives and/or cosmetically acceptable salts thereof. In a preferred embodiment, the solid composition does not comprise a thickener. Excellent viscosities can be achieved upon dilution of the solid composition with water and a viscous liquid with acidic pH is formed, even in the absence of a thickener. In an embodiment, the solid composition of the present invention forms a viscous liquid cleanser with acidic pH upon dilution with water.

The present invention further relates to an aqueous liquid comprising the solid composition of the present invention and an aqueous solvent, wherein the aqueous liquid has a viscosity of 200 cP to 25,000 cP. It is preferred that the viscosity referred to of 200 cP to 25,000 cP is obtained in the absence of a thickener. The viscosity may be even higher by using a thickener. The aqueous liquid is a liquid cleanser. Preferably, the aqueous liquid has a viscosity of 1000 cP to 15,000 cP, more preferably of 1500 cP to 10,000 cP. Again, it is preferred that the aqueous liquid achieves said viscosity in the absence of a thickener. The viscosity may be even higher by using a thickener. It has surprisingly been found that a viscous liquid cleanser can be formed with the solid composition of the present invention, even in the absence of a thickener. In particular, the aqueous liquid is viscous with an acidic pH upon dilution with water. The acidic pH can be achieved using an acidifying agent.

In an embodiment, the aqueous liquid comprises 25 wt. % to 99 wt. % of water, preferably 50 wt. % to 95 wt. % of water, more preferably 75 wt. % to 92 wt. % of water.

In an embodiment, the aqueous liquid comprises the solid composition and water in ratio of 1:20 to 20:1, preferably of 1:15 to 5:1 more preferably of 1:10 to 2:1.

Unless otherwise specified, viscosities used herein are measured at 21° C. with a rheometer using a Brookfield viscometer spindle LV-3 (a.k.a. spindle (63)) or LV-4 (a.k.a. spindle (64)) in accordance with DIN 53019-1:2008-09.

It is preferred that the aqueous solvent comprises at least 50 wt. % water, even more preferred the aqueous solvent is water.

In an embodiment, the aqueous liquid has a pH value of 7 or less, preferably of 4 to 6. The pH value is not particularly limited, but for cosmetic products and skin cleansers, a slightly acidic pH value is preferred.

The present invention further relates to a process of forming the afore-mentioned aqueous liquid, the process comprising mixing the solid composition with an aqueous solvent. It is preferred that the mixing step takes place for 10 to 60 seconds, preferably for 15 to 30 seconds. The mixing step takes place at gentle to moderate mixing rates. It has surprisingly been found that excellent viscosities and uniform clear liquids without lumps can also be achieved when the mixing time is as low as 10 seconds using a gentle mixing rate. Thus in an even more preferred embodiment, the mixing step takes place for less than 1 minute.

The present invention also relates to the use of the solid composition for forming the aqueous liquid.

### EXAMPLES

Examples 1 to 6 were provided as solid compositions containing the components as per Table 1. Sodium C₁₄ alpha olefin sulphonate is obtainable under the trademark Rhodacal® LSS 92 MRP. Cocamidopropyl hydroxysultaine is obtainable under the trademark Mirataine® CBS MB. Guar Hydroxypropyltrimonium Chloride is obtainable under the trademark Jaguar® SOFT.

The solid compositions were subsequently mixed with pure water in the respective amount shown in Table 1 to obtain an aqueous liquid, and thereafter, the viscosity, pH value and appearance of the aqueous liquid were determined. The pH value was determined using a pH-meter. Prior to mixing with water, the solid compositions were dried in a desiccator at ambient temperature to remove any excess moisture (i.e., water).

The viscosity was determined at 25° C. with Brookfield rheometer using a LV-3 spindle (a.k.a. spindle (63)) and following DIN 53019-1:2008-09. As can be seen, the solid compositions of Examples 1 to 6 lead to a clear (Examples 1 to 5) or almost clear (Example 6) aqueous liquid with excellent structure and viscosity.

TABLE 1
Examples 1 to 6*

| | 1 | 2 | 3 | 4 | 5 | 6 |
|---|---|---|---|---|---|---|
| Sodium C14 alpha olefin | 9.16 | 9.01 | 9.06 | 9.56 | 10.21 | 10.36 |
| sulphonate | | | | | | |
| Sodium Cocoyl Glycinate | — | — | — | — | — | 2 |
| 42 wt. % Cocamidopropyl | 6 | 6 | 6 | 6 | 6 | 6 |
| Hydroxysultaine in water | | | | | | |
| Guar | — | 0.5 | 0.5 | — | — | — |
| Hydroxypropyltrimonium | | | | | | |
| Chloride | | | | | | |
| EDTA 2Na | 0.05 | 0.05 | 0.05 | 0.05 | 0.05 | 0.05 |
| Sodium Benzoate | 0.4 | 0.4 | 0.4 | 0.4 | 0.4 | 0.4 |
| Potassium Sorbate | 0.2 | 0.2 | 0.2 | 0.2 | 0.2 | 0.2 |
| Citric acid anhydrous | 1.7 | 1.35 | 1.3 | 1.3 | 1.3 | 0.95 |
| Sodium Chloride | 0.65 | 0.65 | 0.65 | 0.65 | 0 | 0.65 |
| Potassium Ricinoleate | — | — | 1.5 | 1.5 | 1.5 | — |
| Potassium Cocoate | 1.5 | 1.5 | | | | 0.8 |
| Total of solid composition | 19.66 | 19.66 | 19.66 | 19.66 | 19.66 | 21.41 |
| (prior to drying) | | | | | | |
| Total of solid composition | 16.66 | 16.66 | 16.66 | 16.66 | 16.66 | 18.41 |
| (post drying) | | | | | | |
| Diluted with DM water | 83.34 | 83.34 | 83.34 | 83.34 | 83.34 | 81.59 |
| Dilution Factor | 6 | 6 | 6 | 6 | 6 | 5.43 |
| pH | 4.5 | 4.5 | 4.5 | 4.5 | 4.5 | 4.5 |
| Appearance at 25° C.) | Clear | Clear | Clear | Clear | Clear | Translucent |
| | | | | | | with slight |
| | | | | | | turbidity |
| Viscosity in mPa-s using | 3600 | 4908 | 5940 | 3300 | 420 | 6204 |
| LV-3 (S63), RPM-10 at | | | | | | |
| 25° C. | | | | | | |
| Viscosity in mPa-s using | 3469 | 4566 | 5394 | 3288 | 416 | more |
| LV-3 (S63), RPM-20 at | | | | | | than |
| 25° C. | | | | | | 6000 |

*All amounts of the components are in grams

Comparative Examples 1 and 2 were provided as solid compositions containing the components as per Table 2. The solid compositions were subsequently mixed with pure water in the respective amount shown in Table 2 to obtain an aqueous liquid, and thereafter, the viscosity, pH value and appearance of the aqueous liquid were determined as described above. It can be seen that the solid compositions lacking a metal salt of a free fatty acid fail to provide sufficient structure and viscosity when mixed with water. Prior to mixing with water, the solid compositions were dried in a desiccator at ambient temperature to remove any excess moisture (i.e., water).

TABLE 2
Comparative Examples 1 and 2**

| | 1 | 2 |
|---|---|---|
| Sodium C14 alpha | 11.56 | 10.56 |
| olefin sulphonate | | |
| Sodium Cocoyl | 0 | 0 |
| Glycinate | | |
| 42 wt. % | 6 | 8 |
| Cocamidopropyl | | |
| Hydroxysultaine in | | |
| water | | |
| Guar | — | — |
| Hydroxypropyltrimonium | | |
| Chloride | | |
| EDTA 2Na | 0.05 | 0.05 |
| Sodium Benzoate | 0.4 | 0.4 |
| Potassium Sorbate | 0.2 | 0.2 |
| Citric acid anhydrous | 0.8 | 0.8 |
| Sodium Chloride | 0.65 | 0.65 |
| Potassium Ricinoleate | — | — |
| Potassium Cocoate | — | — |
| Total of solid composition | 19.66 | 20.66 |
| (prior to drying) | | |
| Total of solid composition | 16.66 | 16.66 |
| (post drying) | | |
| Dilution with DM water | 83.34 | 83.34 |
| Dilution factor | 6 | 6 |
| pH | 4.5 | 4.5 |
| Appearance at 25° C.) | Clear | Clear |
| Viscosity in mPa-s | 48 | 48 |
| using LV-3 (S63), | | |
| RPM-10 at 25° C. | | |
| Viscosity in mPa-s | 48 | 48 |
| using LV-3 (S63), | | |
| RPM-20 at 25° C. | | |

**All amounts of the components are in grams

Examples 7 to 15 and Comparative Examples 3 to 5 were provided as solid compositions containing the components as per Table 3. The solid compositions were subsequently mixed with pure water in the respective amount shown in Table 3 to obtain an aqueous liquid, and thereafter, the viscosity and pH value of the aqueous liquid were determined as described above.

It can be seen that excellent viscosities can be achieved using a solid composition comprising an anionic surfactant, acyl taurate (oleoyl taurate), and an alkali metal salt of a free fatty acid. In addition, it can be seen that the ratio of the anionic surfactant to the acyl taurate as well as the ratio of the anionic surfactant to the alkali metal salt of a free fatty acid influences the viscosity of the formulation when diluted in water.

Furthermore, the viscosity of the formulation when diluted with water can be further increased by the amount of electrolyte, such as NaCl.

TABLE 3
Examples 7 to 15 and Comparative Examples 3 to 5 (“E” denotes Examples and “CE” denotes Comparative Example)***

| | CE3 | E7 | E8 | E9 | E10 | CE4 | CE5 | E11 | E12 | E13 | E14 | E15 |
|---|---|---|---|---|---|---|---|---|---|---|---|---|
| Sodium C14 alpha olefin sulphonate | 12.06 | 9.06 | 7.01 | 7.29 | 7.14 | 7.01 | | | | | | |
| Sodium Methyl Cocoyl Taurate | | | | | | 5 | 12.01 | 7.01 | 5.7 | 5.7 | 5.7 | 5.45 |
| Sodium Methyl Oleoyl Taurate | 0 | 3 | 5 | 5.32 | 5.32 | 0 | 0 | 5 | 5.7 | 5.7 | 5.7 | 5.45 |
| (75 wt. % active) | | | | | | | | | | | | |
| Guar Hydroxypropyltrimonium | 0.5 | 0.5 | 0.5 | 0.3 | 0.3 | 0.5 | 0.3 | 0.3 | 0.3 | 0.5 | 0.5 | 0.5 |
| Chloride | | | | | | | | | | | | |
| Potassium Ricinoleate | 1.5 | 1.5 | 1.5 | 1.5 | 1.5 | 1.5 | 1.5 | 1.5 | 1.42 | 1.42 | 1.5 | 2 |
| EDTA 2Na | 0.05 | 0.05 | 0.05 | 0.05 | 0.05 | 0.05 | 0.05 | 0.05 | 0.05 | 0.05 | 0.05 | 0.05 |
| Sodium Benzoate | 0.4 | 0.4 | 0.4 | 0.4 | 0.4 | 0.4 | 0.4 | 0.4 | 0.4 | 0.4 | 0.4 | 0.4 |
| Potassium Sorbate | 0.2 | 0.2 | 0.2 | 0.2 | 0.2 | 0.2 | 0.2 | 0.2 | 0.2 | 0.2 | 0.2 | 0.2 |
| Citric acid anhydrous | 1.3 | 1.3 | 1.35 | 0.95 | 0.95 | 1.35 | 1.55 | 1.55 | 1.65 | 1.65 | 1.6 | 2.1 |
| NaCl | 0.65 | 0.65 | 0.65 | 0.65 | 0.8 | 0.65 | 0.65 | 0.65 | 1.24 | 1.04 | 1.01 | 0.51 |
| Total (post drying) | 16.66 | 16.66 | 16.66 | 16.66 | 16.66 | 16.66 | 16.66 | 16.66 | 16.66 | 16.66 | 16.66 | 16.66 |
| Dilution with DM water (quantity) | 83.34 | 83.34 | 83.34 | 83.34 | 83.34 | 83.34 | 83.34 | 83.34 | 83.34 | 83.34 | 83.34 | 83.34 |
| Dilution factor | 6 | 6 | 6 | 6 | 6 | 6 | 6 | 6 | 6 | 6 | 6 | 6 |
| pH | 4.5 | 4.5 | 4.5 | 5.5 | 5.5 | 4.5 | 4.5 | 4.5 | 4.5 | 4.5 | 4.5 | 4.5 |
| Viscosity in mPa-s using | 58 | 1944 | 6456 | 3576 | 4392 | 52 | 46 | 240 | 2148 | 2484 | 4082 | 6444 |
| spindle LV-3 (S63), | | | | | | | | | | | | |
| RPM 10, at 21° C. | | | | | | | | | | | | |

***All amounts of the components are in grams

1. A solid composition comprising:
(a) at least one anionic surfactant,
(b) at least one amphoteric surfactant or at least one acyl taurate, and
(c) at least one alkali metal salt of a free fatty acid,
wherein the wt.-ratio of (a) the at least one anionic surfactant to (b) the at least one amphoteric surfactant or the at least one acyl taurate ranges from 1:3 to 1:0.1, and the wt.-ratio of (a) the at least one anionic surfactant to (c) the at least one alkali metal salt of a free fatty acid ranges from 1:1.5 to 1:0.01, with the proviso that (a) the at least one anionic surfactant and (b) the at least one acyl taurate are different from each other.

2. The solid composition according to claim 1, wherein the composition comprises the at least one anionic surfactant in an amount of 5 wt. % to 80 wt. %, preferably 20 wt. % to 70 wt. %, more preferably 30 wt. % to 60 wt. %.

3. The composition according to claim 1 or 2, wherein the composition comprises the at least one amphoteric surfactant or the at least one acyl taurate in an amount of 5 wt. % to 70 wt. %, preferably 10 wt. % to 60 wt. %, more preferably 12 wt. % to 50 wt. %.

4. The solid composition according to any one of claims 1 to 3, further comprising an electrolyte, preferably an inorganic salt.

5. The solid composition according to claim 4, wherein the electrolyte is an alkali metal salt, preferably selected from NaCl, KCl, Na₂SO₄, K₂SO₄, Na₃PO₄, K₃PO₄, and mixtures of two or more thereof.

6. The solid composition according to claim 4 or 5, wherein the electrolyte is present in an amount of 0.5 wt. % to 30 wt. %, preferably 1 wt. % to 20 wt. %, more preferably 1.5 wt. % to 18 wt. %.

7. The solid composition according to any one of claims 1 to 6, wherein the at least one anionic surfactant is selected from alkyl glycinates, alpha olefin sulphonates, alkyl sulfosuccinates, alkyl sulfo acetates, alkyl lactylates, alkyl carboxylates, alkoxy carboxylates, acyl glycinates, acyl taurate, acyl sarcosinates, alkyl glutamates, acyl glutamates, acyl taurates, acyl sulfates, acyl isethionates, acyl alaninates, and mixtures of two or more thereof; and/or wherein the at least one amphoteric surfactant is selected from alkylamidopropyl betaines, alkyl betaines, alkylamidopropyl hydroxysultainesm alkyl hydroxysultaines, alkyl amphoacetates, alkyl amphodiacetates, alkyl amphopropionates, alkyl amphodipropionates, alkyl amine N-oxides, alkyldimethylamine N-oxides, alkylamidopropylamine N-oxides, and mixtures of two or more thereof, and/or
wherein the at least one alkali metal salt of a free fatty acid is selected from alkali metals salts of C₆ to C₂₄ fatty acids, preferably from potassium cocoate, potassium laurate, potassium oleate, potassium ricinoleate, sodium ricinoleate, sodium palmate, sodium palm kernelate, sodium laurate, sodium caprylate, sodium palmitate, potassium stearate, and mixtures of two or more thereof.

8. The solid composition according to any one of claims 1 to 7, wherein the solid composition does not comprise a thickener.

9. The solid composition according to any one of claims 1 to 8, wherein the solid composition further comprises an acidifying agent, preferably citric acid.

10. The solid composition according to any one of claims 1 to 9, wherein the composition further comprises a nonionic surfactant, preferably selected from alkanolamides, fatty alcohol, fatty alcohol ethoxylates, alkylphenol ethoxylates, glycerol fatty acid amides, glycerol fatty acid esters, sorbitol fatty acid esters, and mixtures of two or more thereof.

11. The solid composition according to any one of claims 1 to 10, wherein the solid composition further comprises any one or more of vitamins, flavonoids, terpenes, saccharides, plant extracts, and/or mixtures of two or more thereof, chelating agents, preservatives, emollients, hair conditioners like guar hydroxypropytrimonium chloride and other derivatives of guar gum, Cationic surfactants or alkyl amidopropyl dialkyl amines and other cationic polymers, skin conditioners, humectants, opacifiers, bulking agents (fillers), polymers, fragrants, antioxidants, pro-vitamins, dyes, abrasives, UV absorbers, anti UV agents, pigments, synthetic and natural benefit actives like anti dandruff agent, anti seborrheic dermatitis agents, anti psoriasis agents, anti inflammatory agents, skin colouring agents, hair colouring agents, melanin regulators, skin lightening, bleaching agents, anti wrinkle agents, anti ageing agents, keratolytic agents, refreshing agents, cooling agents, essential oils, antimicrobial agents, and/or hair transformation, such as straightening, weaving, colouring, and bleaching agents.

12. An aqueous liquid comprising the ingredients of the solid composition according to any one of claims 1 to 11 and an aqueous solvent, wherein the aqueous liquid has a viscosity of 200 cP to 25,000 cP, and wherein the aqueous liquid comprises the solid composition and the aqueous solvent in a wt.-ratio of 1:20 to 20:1, preferably of 1:15 to 5:1 more preferably of 1:10 to 2:1.

13. The aqueous liquid according to claim 12, wherein the aqueous liquid has a pH value of 7 or less, preferably of 4 to 6.5.

14. Use of the solid composition according to any one of claims 1 to 11 for forming the aqueous liquid according to claim 12 or 13.

15. Use of the aqueous liquid according to claim 12 or 13 as a cleaning composition, personal care composition, disinfection composition, floor cleanser, toilet bowl cleanser, glass cleanser, metal cleansing composition and/or preparations thereof.
